(19) United States
(12) Patent Application Publication
(10) Pub. No.: US 2007/0203238 A1
(43) Pub. Date: Aug. 30, 2007

(54) Method for preventing or reducing elevated triglyceride levels

(76) Inventors: Zeina Jouni, Evansville, IN (US); Joshua C. Anthony, Evansville, IN (US); Steven C. Rumsey, Curitiba (BR); Deborah A. Schade, Evansville, IN (US); James T. Brenna, Ithaca, NY (US); Andrea Tseng Hsieh, Ithaca, NY (US)

Correspondence Address: Richard D. Schmidt;Bristol-Myers Squibb Company, 2400 West Lloyd Espressway, Evansville, IN, 47721-0001, US

(21) Appl. No.: 11/711,411

(22) Filed: Feb. 27, 2007

Related U.S. Application Data
(60) Provisional application No. 60/777,334, filed on Feb. 28, 2006.

(51) Int. Cl. A61K 31/202

(52) U.S. Cl. 514/560

(57) ABSTRACT

The present invention is directed to a novel method for reducing triglyceride levels in an infant. The method comprises administration of a therapeutically effective amount of DHA and ARA, alone or in combination with one another, to the subject.

This application claims the priority benefit of U.S. Provisional Application 60/777,334 filed Feb. 28, 2006 which is incorporated by reference herein in its entirety.

### BACKGROUND OF THE INVENTION

(1) Field of the Invention

The present invention relates generally to a method for preventing or reducing elevated triglyceride levels.

(2) Description of the Related Art

Triglycerides (also known as triacylglycerols or triacylglycerides) are glycerides in which the glycerol is esterified with three fatty acids. They are the main constituent of vegetable oil and animal fats. Triglycerides play an important role as energy sources in metabolism because they contain more than twice as much energy as carbohydrates and proteins. In the human intestine, triglycerides are split into glycerol and fatty acids with the help of lipases and bile secretions. The glycerol molecules and fatty acids can then move into the blood vessels. The triglycerides are rebuilt in the blood from their fragments and become constituents of lipoproteins. Various tissues can release the free fatty acids from triglycerides and take them up as a source of energy. Fat cells can also synthesize and store triglycerides. Other than water, the bulk of body fat tissue is in the form of triglycerides. When the body requires fatty acids as an energy source, the hormone glucagon signals the breakdown of the triglycerides by hormone-sensitive lipase to release free fatty acids.

In the human body, however, high levels of triglycerides in the bloodstream have been linked to atherosclerosis, and, by extension, the risk of heart disease and stroke, as well as diabetes mellitus, pancreatitis, chronic renal disease, and certain primary hyperlipidemias. Though the nature of the relationship is unclear, high triglyceride levels have also been associated with obesity. Additionally, high triglyceride levels have been associated with depression, bipolar disorder, and other affective disorders. See Glueck, C. J., et al., Hypocholesterolemia and Affective Disorders, Am. J. Med. Sci. 308(4):218-225 (1994).

Though the precise relationship between high triglyceride levels and these diseases and disorders is still under investigation, most experts recommend taking affirmative steps to lower triglyceride levels. Evidence also shows that sustained aerobic activity can have an impact on blood triglyceride levels. Additionally, omega-3 fatty acids such as docosahexaenoic acid (DHA) and eicosapentaenoic acid (EPA) have been indicated to lower triglyceride levels in adults. For example, the American Heart Association recommends that adults consume 2 to 4 grams of DHA and EPA per day to lower triglyceride levels.

Research has shown that fatty buildups in arteries begin in childhood and are more likely to occur with higher blood cholesterol and triglyceride levels. There is also growing evidence that adult cholesterol levels may be influenced by factors that operate very early in life, even in infancy. Owen, C. G., et al., Infant Feeding and Blood Cholesterol: A Study in Adolescents and a Systematic Review, Pediatr. 110(3):597-608 (2002). For example, low birth weight, bottle feeding and prolonged breast feeding have been associated with higher adult cholesterol levels. Id.

Thus, because it is possible that cholesterol and triglyceride levels in adulthood may be affected by various factors in infancy and childhood, it would be beneficial to maintain normal triglyceride levels in infancy. The maintenance of normal triglyceride levels in infancy may potentially reduce the risk of elevated triglyceride levels in adulthood and prevent the onset of various diseases and disorders.

Recommendations for lowering triglyceride levels in children are dramatically different than that for adults, however. For example, the Committee on Nutrition of the American Academy of Pediatrics recommends that children be screened for cholesterol and triglyceride levels only if: (1) a parent or grandparent had atherosclerosis at or before age 55, (2) a parent or grandparent suffered a heart attack or showed other signs of artery disease at or before age 55, or (3) a parent has a blood cholesterol level over 240.

Regardless of family history, however, infants and children under the age of two that are physically healthy and normal should not be put on a low-fat or low-cholesterol diet. Fats and cholesterol are important for normal growth and development in young children and depriving them of adequate amounts of these substances can be dangerous.

Therefore, it would be beneficial to provide a composition that reduces triglyceride levels in infants without adjusting their dietary intake of fat, glucose-increasing foods, or cholesterol-containing foods. It would also be beneficial to provide a nutritional supplement or infant formula containing such a composition in order to lower triglyceride levels without compromising needs.

### SUMMARY OF THE INVENTION

Briefly, therefore, the present invention is directed to a novel method for reducing triglyceride levels in a subject. The subject may be an infant or child. The method comprises administering a therapeutically effective amount of DHA or ARA, alone or in combination with one another, to the subject. The invention is also directed to a novel method for preventing elevated triglyceride levels in a subject.

Among the several advantages found to be achieved by the present invention, is that the prevention or reduction of triglyceride levels in infancy can provide a reduced likelihood of triglyceride-linked diseases and disorders in childhood, adolescence or adulthood.

### BRIEF DESCRIPTION OF THE DRAWINGS

For a more complete understanding of the present invention, reference is now made to the following descriptions taken in conjunction with the accompanying drawings.

FIG. 1 is a graph illustrating the effects of DHA and ARA supplementation on serum triglyceride levels in neonate baboons.

### DETAILED DESCRIPTION OF THE PREFERRED EMBODIMENTS

Reference now will be made in detail to the embodiments of the invention, one or more examples of which are set forth below. Each example is provided by way of explanation of the invention, not a limitation of the invention. In fact, it will be apparent to those skilled in the art that various modifications and variations can be made in the present invention without departing from the scope or spirit of the invention. For instance, features illustrated or described as part of one embodiment, can be used on another embodiment to yield a still further embodiment.

Thus, it is intended that the present invention covers such modifications and variations as come within the scope of the appended claims and their equivalents. Other objects, features and aspects of the present invention are disclosed in or are obvious from the following detailed description. It is to be understood by one of ordinary skill in the art that the present discussion is a description of exemplary embodiments only, and is not intended as limiting the broader aspects of the present invention.

As used herein, the term “reducing” means bringing down or diminishing the level of triglycerides.

The term “preventing” means to stop or hinder a disease, disorder, or symptom of a disease or condition through some action.

The terms “therapeutically effective amount” refer to an amount that results in an improvement or remediation of the disease, disorder, or symptoms of the disease or condition.

The term “infant” means a postnatal human that is less than about 1 year of age.

The term “child” means a human that is between about 1 year and 12 years of age. In some embodiments, a child is between the ages of about 1 and 6 years. In other embodiments, a child is between the ages of about 7 and 12 years.

As used herein, the term “infant formula” means a composition that satisfies the nutrient requirements of an infant by being a substitute for human milk. In the United States, the contents of an infant formula are dictated by the federal regulations set forth at 21 C.F.R. Sections 100, 106, and 107. These regulations define macronutrient, vitamin, mineral, and other ingredient levels in an effort to stimulate the nutritional and other properties of human breast milk.

In accordance with the present invention, the inventors have discovered a novel method for reducing triglyceride levels in infants which comprises administering a therapeutically effective amount of docosahexaenoic acid (DHA) and arachidonic acid (ARA) to the subject. In fact, it has been shown in the present invention that the administration of 0.33% DHA and 0.67% ARA, as a percentage of total fatty acid, can reduce triglyceride levels by as much as about 39%. Additionally, the administration of 1.00% DHA and 0.67% ARA can reduce triglyceride levels by as much as about 24%.

DHA and ARA are long chain polyunsaturated fatty acids (LCPUFA) which have been shown to contribute to the health and growth of infants. Specifically, DHA and ARA have been shown to support the development and maintenance of the brain, eyes and nerves of infants. Birch, E., et al., A Randomized Controlled Trial of Long-Chain Polyunsaturated Fatty Acid Supplementation of Formula in Term Infants after Weaning at 6 Weeks of Age, Am. J. Clin. Nutr. 75:570-580 (2002). Clandinin M., et al., Growth and Development of Preterm Infants Fed Infant Formulas Containing Docosahexaenoic Acid and Arachidonic Acid, J. Pediatr. 146(4): 461-8 (2005). DHA and ARA are typically obtained through breast milk in infants that are breast-fed. In infants that are formula-fed, however, DHA and ARA must be supplemented into the diet.

While it has been shown that DHA and ARA are beneficial to the development of brain, eyes and nerves in infants, DHA and ARA have not previously been shown to have any effect on triglycerides levels in infants. The positive effects of DHA and ARA on triglyceride levels in infants that were discovered in the present invention were surprising and unexpected.

In certain embodiments of the present invention, the subject is in need of a reduction of triglyceride levels or a prevention of elevated triglyceride levels. In this embodiment, the subject can be an subject that is at risk for having high triglyceride levels or may already have high triglyceride levels. The subject can be at risk due to genetic predisposition, inherited disorders, diet, diseases or disorders, and the like. For example, the subject may be at risk for developing atherosclerosis, heart disease, diabetes mellitus, pancreatitis, chronic renal disease, certain primary hyperlipidemias, obesity, depression, bipolar disorder or other affective disorders. As another example, the subject could be at risk because a parent or grandparent had atherosclerosis at or before age 55, a parent or grandparent suffered a heart attack or showed other signs of artery disease at or before age 55, or a parent has a blood cholesterol level over 240.

In the present invention, the form of administration of DHA and ARA is not critical, as long as a therapeutically effective amount is administered to the subject. In some embodiments, the DHA and ARA are administered to a subject via tablets, pills, encapsulations, caplets, gelcaps, capsules, oil drops, or sachets. In another embodiment, the DHA and ARA are added to a food or drink product and consumed. The food or drink product may be a children's nutritional product such as a follow-on formula, growing up milk, or a milk powder or the product may be an infant's nutritional product, such as an infant formula.

In an embodiment, the infant formula for use in the present invention is nutritionally complete and contains suitable types and amounts of lipid, carbohydrate, protein, vitamins and minerals. The amount of lipid or fat typically can vary from about 3 to about 7 g/100 kcal. The amount of protein typically can vary from about 1 to about 5 g/100 kcal. The amount of carbohydrate typically can vary from about 8 to about 12 g/100 kcal. Protein sources can be any used in the art, e.g., nonfat milk, whey protein, casein, soy protein, hydrolyzed protein, amino acids, and the like. Carbohydrate sources can be any used in the art, e.g., lactose, glucose, corn syrup solids, maltodextrins, sucrose, starch, rice syrup solids, and the like. Lipid sources can be any used in the art, e.g., vegetable oils such as palm oil, canola oil, corn oil, soybean oil, palmolein, coconut oil, medium chain triglyceride oil, high oleic sunflower oil, high oleic safflower oil, and the like.

Conveniently, commercially available infant formula can be used. For example, Enfalac, Enfamil®, Enfamil® Premature Formula, Enfamil® with Iron, Lactofree®, Nutramigen®, Pregestimil®, and ProSobee® (available from Mead Johnson & Company, Evansville, Ind., U.S.A.) may be supplemented with suitable levels of DHA or ARA, alone or in combination with one another, and used in practice of the method of the invention. Additionally, Enfamil® LIPIL®, which contains effective levels of DHA and ARA, is commercially available and may be utilized in the present invention.

The method of the invention requires the administration of DHA or ARA, alone or in combination with one another. In this embodiment, the weight ratio of ARA:DHA is typically from about 1:3 to about 9:1. In one embodiment of the present invention, this ratio is from about 1:2 to about 4:1. In yet another embodiment, the ratio is from about 2:3 to about 2:1. In one particular embodiment the ratio is about 2:1. In another particular embodiment of the invention, the ratio is about 1:1.5. In other embodiments, the ratio is about 1:1.3. In still other embodiments, the ratio is about 1:1.9. In a particular embodiment, the ratio is about 1.5:1. In a further embodiment, the ratio is about 1.47:1.

In certain embodiments of the invention, the level of DHA is between about 0.0% and 1.00% of fatty acids, by weight. Thus, in certain embodiments, the ARA alone may reduce triglyceride levels.

The level of DHA may be about 0.32% by weight. In some embodiments, the level of DHA may be about 0.33% by weight. In another embodiment, the level of DHA may be about 0.64% by weight. In another embodiment, the level of DHA may be about 0.67% by weight. In yet another embodiment, the level of DHA may be about 0.96% by weight. In a further embodiment, the level of DHA may be about 1.00% by weight.

In embodiments of the invention, the level of ARA is between 0.0% and 0.67% of fatty acids, by weight. Thus, in certain embodiments of the invention, DHA alone can reduce triglyceride levels. In another embodiment, the level of ARA may be about 0.67% by weight. In another embodiment, the level of ARA may be about 0.5% by weight. In yet another embodiment, the level of DHA may be between about 0.47% and 0.48% by weight.

The effective amount of DHA in an embodiment of the present invention is typically from about 3 mg per kg of body weight per day to about 150 mg per kg of body weight per day. In one embodiment of the invention, the amount is from about 6 mg per kg of body weight per day to about 100 mg per kg of body weight per day. In another embodiment the amount is from about 15 mg per kg of body weight per day to about 60 mg per kg of body weight per day.

The effective amount of ARA in an embodiment of the present invention is typically from about 5 mg per kg of body weight per day to about 150 mg per kg of body weight per day. In one embodiment of this invention, the amount varies from about 10 mg per kg of body weight per day to about 120 mg per kg of body weight per day. In another embodiment, the amount varies from about 15 mg per kg of body weight per day to about 90 mg per kg of body weight per day. In yet another embodiment, the amount varies from about 20 mg per kg of body weight per day to about 60 mg per kg of body weight per day.

The amount of DHA in infant formulas for use in the present invention typically varies from about 2 mg/100 kilocalories (kcal) to about 100 mg/100 kcal. In another embodiment, the amount of DHA varies from about 5 mg/100 kcal to about 75 mg/100 kcal. In yet another embodiment, the amount of DHA varies from about 15 mg/100 kcal to about 60 mg/100 kcal.

The amount of ARA in infant formulas for use in the present invention typically varies from about 4 mg/100 kilocalories (kcal) to about 100 mg/100 kcal. In another embodiment, the amount of ARA varies from about 10 mg/100 kcal to about 67 mg/100 kcal. In yet another embodiment, the amount of ARA varies from about 20 mg/100 kcal to about 50 mg/100 kcal. In a particular embodiment, the amount of ARA varies from about 25 mg/100 kcal to about 40 mg/100 kcal. In one embodiment, the amount of ARA is about 30 mg/100 kcal.

The infant formula supplemented with oils containing DHA and ARA for use in the present invention can be made using standard techniques known in the art. For example, replacing an equivalent amount of an oil normally present, e.g., high oleic sunflower oil.

The source of the ARA and DHA can be any source known in the art such as marine oil, fish oil, single cell oil, egg yolk lipid, brain lipid, and the like. The DHA and ARA can be in natural form, provided that the remainder of the LCPUFA source does not result in any substantial deleterious effect on the infant. Alternatively, the DHA and ARA can be used in refined form.

In one embodiment, the LCPUFA source contains eicosapentaenoic acid (EPA). In another embodiment, the LCPUFA source is substantially free of EPA. For example, in one embodiment, the infant formulas used herein contain less than about 20 mg/100 kcal EPA; in another embodiment, less than about 10 mg/100 kcal EPA; in still another embodiment, less than about 5 mg/100 kcal EPA; and in a particular embodiment, substantially no EPA.

Sources of DHA and ARA may include single cell oils as taught in U.S. Pat. Nos. 5,374,657, 5,550,156, and 5,397,591, the disclosures of which are incorporated herein by reference in their entirety.

In an embodiment of the present invention, DHA or ARA, alone or in combination with one another, are supplemented into the diet of an infant from birth until the infant reaches about one year of age. In a particular embodiment, the infant can be a preterm infant. In another embodiment of the invention, DHA or ARA, alone or in combination with one another, are supplemented into the diet of a subject from birth until the subject reaches about two years of age. In other embodiments, DHA or ARA, alone or in combination with one another, are supplemented into the diet of a subject for the lifetime of the subject. Thus, in particular embodiments, the subject may be a child, adolescent, or adult.

In an embodiment, the subject of the invention is a child between the ages of one and six years old. In another embodiment the subject of the invention is a child between the ages of seven and twelve years old. In particular embodiments, the administration of DHA to children between the ages of one and twelve years of age is effective in reducing triglyceride levels. In other embodiments, the administration of DHA and ARA to children between the ages of one and twelve years of age is effective in reducing triglyceride levels.

In the present invention, DHA or ARA, alone or in combination with one another, supplementation is effective in treating or preventing atherosclerosis, heart disease, diabetes mellitus, pancreatitis, chronic renal disease, certain primary hyperlipidemias, obesity, depression, bipolar disorder or other affective disorders.

Though not wishing to be bound to this or any theory, the mechanism of action in the present invention could range from increasing clearance of triglyceride-rich lipoproteins (chylomicrons and very low density lipoprotein), decreasing the synthesis of triglyceride-rich lipoproteins, increasing utilization of triglyceride, activating peroxisome-proliferator activated receptors, and/or increasing beta-oxidation of fatty acids in muscle cells and hepatocytes.

Although omega-3 fatty acids such as DHA and EPA have previously been indicated to lower triglyceride levels in adults, these LCPUFAs have not been suggested for lowering triglyceride levels in infants. Additionally, the present invention utilizes the specific combination of DHA or ARA, alone or in combination with one another, for lowering triglyceride levels in infants.

The present invention is also directed to the use of DHA or ARA, alone or in combination with one another, for the preparation of a composition or medicament for the lowering of triglyceride levels. In this embodiment, the DHA or ARA, alone or in combination with one another, may be used to prepare a medicament for the lowering of triglyceride levels in any human or animal subject. For example, the medicament could be used to lower triglyceride levels in domestic, farm, zoo, sports, or pet animals, such as dogs, horses, cats, cattle, and the like. In some embodiments, the animal is in need of the lowering of triglyceride levels.

The following examples describe various embodiments of the present invention. Other embodiments within the scope of the claims herein will be apparent to one skilled in the art from consideration of the specification or practice of the invention as disclosed herein. It is intended that the specification, together with the examples, be considered to be exemplary only, with the scope and spirit of the invention being indicated by the claims which follow the examples. In the examples, all percentages are given on a weight basis unless otherwise indicated.

### EXAMPLE 1

This example illustrates the influence of zero, moderate, and high levels of DHA on serum triglyceride in term baboons from 2 to 12 weeks of age.

### Methods

### Animals

All animal work took place at the Southwest Foundation for Biomedical Research (SFBR) located in San Antonio, Tex. Animal protocols were approved by the SFBR and Cornell University Institutional Animal Care and Use Committee (IACUC). Animal characteristics are summarized in Table 1.

TABLE 1
Baboon Neonate Characteristics

| | |
|---|---|
| Number of animals | 14 |
| Gender | 10 female, 4 male |
| Conceptional age at delivery (days) | 181.8 ± 6.2 |
| Birth weight (g) | 860.3 ± 150.8 |
| Weight at 12 weeks (g) | 1519.1 ± 280.7 |
| Weight gain (g) | 658.8 ± 190.4 |

Fourteen pregnant baboons delivered spontaneously around 182 days gestation. Neonates were transferred to the nursery within 24 hours of birth and randomized to one of three diet groups. Animals were housed in enclosed incubators until 2 weeks of age and then moved to individual stainless steel cages in a controlled access nursery. Room temperatures were maintained at temperatures between 76° F. to 82° F., with a 12 hour light/dark cycle. They were fed on experimental formulas until 12 weeks of life.

### Diets

Animals were assigned to one of the three experimental formulas, with LCPUFA concentrations presented in Table 2.

TABLE 2
Formula LCPUFA composition

| | C | L | L3 |
|---|---|---|---|
| DHA (%, w/w) | 0 | 0.42 ± 0.02 | 1.13 ± 0.04 |
| DHA | 0 | 21.3 ± 1.0 | 62.8 ± 1.9 |
| (mg/100 kcal) | | | |
| ARA (%, w/w) | 0 | 0.77 ± 0.02 | 0.71 ± 0.01 |
| ARA (mg/100 kcal) | 0 | 39.4 ± 0.9 | 39.2 ± 0.7 |

Target concentrations were set as shown in brackets and diets were formulated with excess to account for analytical and manufacturing variability and/or possible losses during storage. Control (C) and L, moderate DHA formula, are the commercially available human infant formulas Enfamil® and Enfamil LIPIL®, respectively. Formula L3 had an equivalent concentration of ARA and was targeted at three-fold the concentration of DHA.

Formulas were provided by Mead Johnson & Company (Evansville, Ind.) in ready-to-feed form. Each diet was sealed in cans assigned two different color-codes to mask investigators. Animals were offered 1 ounce of formula four times daily at 07:00, 10:00, 13:00 and 16:00 with an additional feed during the first 2 nights. On day 3 and beyond, neonates were offered 4 ounces total; when they consumed the entire amount, the amount offered was increased in daily 2 ounce increments. Neonates were hand fed for the first 7-10 days until independent feeding was established.

### Blood Sampling

Blood was obtained via femoral venipuncture in fasted animals between 07:00 and 08:30. One mL blood samples were obtained from neonates weighing less than 1 kg; 1.5 mL was drawn from animals weighing between 1 and 1.5 kg. Serum clinical chemistries were assessed at 6 and 12 weeks of age. White cell measurements were made on whole blood collected in potassium ethylenediaminetetraacetic acid (EDTA) microtainer tubes at 2, 4, 8, 10 and 12 weeks of age.

### Clinical Chemistry and White Cell Measurements

All samples were analyzed at the Clinical Pathology Laboratory at the Southwest Foundation for Biomedical Research. Variables evaluated were glucose, blood urea nitrogen (BUN), creatinine, total protein, albumin, globulin, albumin/globulin ratio (A/G ratio), cholesterol, serum glutamine-pyruvate transaminase (SGPT), serum glutamic-oxaloacetic transaminase (SGOT), alkaline phosphatase, sodium, potassium, chloride, carbon dioxide, anion gap, gamma glutamyl transferase (GGT), lactate dehydrogenase (LDH), creatine phosphokinase (CPK), total bilirubin, direct bilirubin, calcium, phosphorus, and triglycerides. Analyses were preformed using a Beckman Synchron CX5CE (Beckman Coulter, Inc., Fullerton, Calif.). Determination details have been reported previously. CBC parameters were white blood cell (WBC) counts, platelet count, mean platelet volumes (MPV), neutrophils, lymphocytes, monocytes, eosinophils, and basophils. Red cell parameters were significantly related to DHA/ARA levels and are the subject of a separate report. Measurements were determined using a Coulter MAXM autoloader instrument (Beckman Coulter, Inc., Fullerton, Calif.).

### Statistics

Data are expressed as mean±SD. Statistical analyses for biochemistry values were conducted using repeated measures ANOVA, with diet treatment (C, L, L3) as a between-group factor and age (6, 12) as within group factors. White cell values were evaluated using a random coefficient regression model to examine systematic effects of diet over time. Analyses were performed using SAS for Windows 9.1 (SAS Institute, Cary, N.C.) with significance declared at p<0.05.

### Results

### Clinical Chemistry

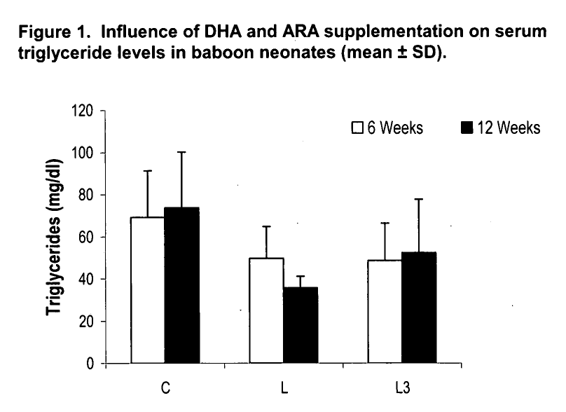

FIGS. 1 and 2 present the results for the two parameters that were significantly influenced by the different infant formulas. At six weeks, significant differences due to dietary LCPUFAs were seen in serum triglyceride values (TG) (FIG. 1). TG values were significantly influenced by diet; C levels were higher than both LCPUFA groups (p=0.03). Mean TG values were 71.8±23.3 for the control group and 43.7±13.5 (L) and 54.7±20.2 (L3) for LCPUFA animals. Between 6 and 12 weeks of age, no changes were detected. Table 3 summarizes the biochemical data obtained at 6 and 12 weeks of age.

TABLE 3
Ontogeny of Clinical Chemistry Parameters for Baboon
Neonates (mean ± SD, range)

| Parameter (unit) | 6 Weeks | 12 Weeks |
|---|---|---|
| Glucose (mg/dl) | 56.4 ± 14.4, 36–68 | 76.5 ± 15.6, 63–112 |
| Creatinine (mg/dl) | 0.6 ± 0.1, 0.3–0.7 | 0.5 ± 0.1, 0.3–0.7 |
| Total Protein (g/dl) | 6.0 ± 0.5, 5.6–6.3 | 5.2 ± 0.3, 4.8–5.2 |
| Globulin (g/dl) | 2.3 ± 0.3, 1.9–2.6 | 1.7 ± 0.3, 1.2–1.9 |
| A/G Ratio | 1.5 ± 0.3, 1.3–1.9 | 2.1 ± 0.4, 1.7–3.1 |
| SGOT (U/l) | 39.8 ± 9.5, 34–44 | 31.7 ± 5.2, 29–43 |
| Potassium (mEq/l) | 4.9 ± 0.4, 4.3–5 | 3.7 ± 0.7, 2.9–5.5 |
| Carbon Dioxide (mEq/l) | 18.4 ± 2.5, 17.22 | 22.9 ± 2.1, 19–26 |
| Anion Gap (mEq/l) | 18.8 ± 2.3, 16.3–21.9 | 10.1 ± 2.8, 7.4–12.5 |
| LDH (U/l) | 288.2 ± 53.3, 235–390 | 251.1 ± 39.7, 225–330 |
| Total Bilirubin (mg/dl) | 0.6 ± 0.1, 0.5–0.7 | 0.4 ± 0.1, 0.3–0.4 |
| BUN (mg/dl) | 8.7 ± 2.1, 6–8 | 8.6 ± 2.2, 7–10 |
| BUN/Creatinine Ratio | 14.7 ± 3.5, 8.6–14 | 19.0 ± 5.1, 14–25 |
| Albumin (g/dl) | 3.6 ± 0.1, 3.4–3.7 | 3.5 ± 0.2, 3.2–3.7 |
| Cholesterol (mg/dl) | 94.6 ± 14.7, 92–123 | 95.0 ± 15.7, 72–124 |
| SGPT (U/l) | 25.5 ± 10.4, 15–21 | 27.6 ± 8.9, 17–29 |
| Alkaline phosphatase | 1304.5 ± 191.3, | 1264.0 ± 234.6, |
| (U/l) | 981–1552 | 849–1782 |
| Sodium (mEq/l) | 144.2 ± 2.0, 142–146 | 144.5 ± 1.5, 144–147 |
| Chloride (mEq/l) | 111.9 ± 1.9, 109–112 | 115.4 ± 1.8, 114–118 |
| GGT (U/l) | 70.5 ± 15.7, 43–99 | 65.5 ± 14.4, 42–84 |
| Direct Bilirubin (mg/dl) | 0.1 ± 0.1, 0.1–0.2 | 0.1 ± 0.0, 0.1–0.2 |
| CPK (U/l) | 186.8 ± 64.7, 96–323 | 445.3 ± 212.2, |
| | | 273–885 |
| Phosphorus (mg/dl) | 7.6 ± 0.7, 7.8–8 | 7.8 ± 0.7, 6.4–9.1 |

Neonatal baboon measurements for serum GGT, LDH, total bilirubin, direct bilirubin, CPK, calcium, phosphorus and triglycerides have not been reported previously. Mean values for those parameters for which there are literature values are similar to present values. Mean values for serum glucose, A/G ratio, and carbon dioxide increased from 6 to 12 weeks. Means for creatinine, total protein, globulin, SGOT, potassium, anion gap, LDH and total bilirubin values decreased significantly from 6 to 12 weeks of age. No change was detected between the two time points for BUN, BUN/creatinine ratio, albumin, cholesterol, SGPT, alkaline phosphatase, sodium, chloride, GGT, direct bilirubin, CPK, and phosphorus levels.

### White Cell Measurements

Results for white cell measurements are presented in Table 4.

TABLE 4
White cell parameters ontogeny for baboon neonates (mean ± SD)

| Parameter | Age (weeks) | | | | |
|---|---|---|---|---|---|
| (Units) | 2 | 4 | 8 | 10 | 12 |
| Basophil (%) | 0.97 ± 2.6 | 0.49 ± 0.98 | 0.25 ± 0.44 | 0.30 ± 0.47 | 0.09 ± 0.13 |
| Monocyte (%) | 2.4 ± 1.6 | 2.2 ± 1.4 | 4.6 ± 2.6 | 4.4 ± 1.8 | 4.5 ± 1.3 |
| Platelet Count | 416 ± 133 | 350 ± 141 | 314 ± 88 | 286 ± 78 | 362 ± 99 |
| (×10³) | | | | | |
| WBC (×10³) | 6.9 ± 1.4 | 9.00 ± 1.9 | 8.8 ± 2.0 | 8.5 ± 2.2 | 5.2 ± 1.7 |
| MPV (fl) | 8.5 ± 0.7 | 8.7 ± 0.7 | 8.6 ± 0.8 | 8.8 ± 0.9 | 7.7 ± 0.6 |
| Neutrophils | 39 ± 20 | 40 ± 12 | 28 ± 10 | 20 ± 7 | 34 ± 9 |
| (%) | | | | | |
| Lymphocyte | 54 ± 19 | 54 ± 10 | 65 ± 10 | 73 ± 9 | 60 ± 9 |
| (%) | | | | | |
| Eosinophil | 1.4 ± 1.1 | 1.8 ± 1.0 | 2.4 ± 1.2 | 1.4 ± 0.6 | 1.0 ± 0.5 |
| (%) | | | | | |

Although dietary DHA and ARA caused changes in RBC, hemoglobin, hematocrit and RDW, no effects of LCPUFA were found for the white cell parameters. Age-related changes were seen for basophils and monocytes. Significantly decreasing values were observed for basophils percentages. Monocytes, however, increased in baboon neonates approximately 45% during the first 12 weeks after birth.

### Discussion

In the present invention, LCPUFA consumption significantly influenced serum triglyceride measurements in baboon neonates. Triglyceride levels were significantly lower for LCPUFA-fed neonates compared to controls consuming formula devoid of LCPUFAs.

Longitudinal changes in serum clinical chemistry parameters (glucose, creatinine, total protein, globulin, A/G ratio, SGOT, potassium, carbon dioxide, and anion gap) were within published ranges for baboons. Decreasing LDH and total bilirubin values have not previously been reported for baboons. Their decrease is consistent with changes in human infants and may indicate hepatic maturation. Serum glucose and carbon dioxide in healthy human infants increases from birth to 12 weeks of age, and are consistent with the present findings. Although baboon albumin values did not change, the albumin/globulin ratio (A/G ratio) increased from 6 to 12 weeks of age due to decreasing serum globulin concentrations observed in the animals, similar to human neonates.

Most of the comparisons of the data to reference values, where available, show trends consistent with patterns seen in normal, healthy term baboon and human infant hematological development. During the first weeks after birth, significant decreases in basophils were observed, while monocytes percentages increased.

This is the first study examining the effects of DHA and ARA consumption on biochemical and white cell parameters of baboon neonates. The effects of increasing levels of dietary LCPUFA from 2 to 12 weeks of age were evaluated. Consumption of 0.33% DHA/0.67% ARA and 1.00% DHA/0.67% ARA significantly influenced triglyceride levels when compared to a control group consuming LCPUFA-free formula. Neither set of values were outside the normal ranges for this age group. Overall, white cell values were similar to established infant baboon reference ranges and consistent with trends observed during human postnatal development.

All references cited in this specification, including without limitation, all papers, publications, patents, patent applications, presentations, texts, reports, manuscripts, brochures, books, internet postings, journal articles, periodicals, and the like, are hereby incorporated by reference into this specification in their entireties. The discussion of the references herein is intended merely to summarize the assertions made by their authors and no admission is made that any reference constitutes prior art. Applicants reserve the right to challenge the accuracy and pertinence of the cited references.

Although preferred embodiments of the invention have been described using specific terms, devices, and methods, such description is for illustrative purposes only. The words used are words of description rather than of limitation. It is to be understood that changes and variations may be made by those of ordinary skill in the art without departing from the spirit or the scope of the present invention, which is set forth in the following claims. In addition, it should be understood that aspects of the various embodiments may be interchanged both in whole or in part. For example, while methods for the production of a commercially sterile liquid nutritional supplement made according to those methods have been exemplified, other uses are contemplated. Therefore, the spirit and scope of the appended claims should not be limited to the description of the preferred versions contained therein.

What is claimed is:

1. A method for reducing triglyceride levels in an infant, the method comprising administering to the infant a therapeutically effective amount of DHA and ARA.

2. The method according to claim 1, wherein the infant is in need of such reduction.

3. The method according to claim 1, wherein the therapeutically effective amount of DHA is between about 15 mg per kg of body weight per day and 60 mg per kg of body weight per day.

4. The method according to claim 1, wherein the therapeutically effective amount of ARA is between about 20 mg per kg of body weight per day and 60 mg per kg of body weight per day.

5. The method according to claim 1, wherein the ratio of ARA:DHA by weight is from about 1:3 to about 9:1.

6. The method according to claim 1, wherein the ratio of ARA:DHA by weight is about 2:1.

7. The method according to claim 1, wherein the ratio of ARA:DHA by weight is about 1:1.5.

8. The method according to claim 1, wherein DHA comprises between about 0.33% and 1.00% of fatty acids by weight.

9. The method according to claim 1, wherein the DHA and ARA are administered to the infant during the time period from birth until the infant is about one year of age.

10. The method according to claim 1, wherein the DHA and ARA are administered to the infant in an infant formula.

11. The method according to claim 10 wherein the infant formula comprises DHA in an amount of from about 15 mg to about 60 mg per 100 kcal infant formula.

12. The method according to claim 10 wherein the infant formula comprises ARA in an amount of from about 25 mg to about 40 mg per 100 kcal infant formula.

13. A method for preventing elevated triglyceride levels in an infant, the method comprising administering to the infant a therapeutically effective amount of DHA and ARA.

14. The method according to claim 13 wherein the infant is in need of such prevention.

15. A method for reducing triglyceride levels in an infant, the method comprising administering to the infant a therapeutically effective amount of DHA and ARA, wherein the ratio of ARA:DHA by weight is about 1:1.5.

16. A method for reducing triglyceride levels in an infant, the method comprising administering to the infant a therapeutically effective amount of DHA and ARA, wherein DHA comprises between about 0.33% and 1.00% of fatty acids by weight.

17. A method for reducing triglyceride levels in an infant, the method comprising administering to the infant a therapeutically effective amount of DHA.

18. The method according to claim 17, wherein DHA comprises between about 0.33% and 1.00% of fatty acids by weight.

19. A method for reducing triglyceride levels in an infant, the method comprising administering to the infant a therapeutically effective amount of ARA.

20. A method for reducing triglyceride levels in a child, the method comprising administering to the child DHA.

21. The method according to claim 20, wherein the child is between the ages of one and six years of age.

22. The method according to claim 20, wherein the child is between the ages of about seven and twelve years of age.

23. The method according to claim 20 additionally comprising administering ARA to the child.

24. A method for reducing triglyceride levels in a child, the method comprising administering to the child ARA.
